(19) United States
(12) Patent Application Publication
(10) Pub. No.: US 2026/0109966 A1
(43) Pub. Date: Apr. 23, 2026

(54) METHODS FOR PREPARING INTERACTION PLASMID LIBRARY AND TESTING INTERACTION BETWEEN cDNA LIBRARIES

(71) Applicant: HuBei Hongshan Laboratory, Wuhan (CN)

(72) Inventors: Lin LI, Wuhan (CN); Xiaoyang SHANG, Wuhan (CN)

(73) Assignee: HuBei Hongshan Laboratory, Wuhan (CN)

(21) Appl. No.: 19/000,135

(22) Filed: Dec. 23, 2024

Related U.S. Application Data
(63) Continuation of application No. PCT/CN2024/133915, filed on Nov. 22, 2024.

(30) Foreign Application Priority Data
Oct. 18, 2024 (CN) 2024114663808

(51) Int. Cl. C12N 15/10; C12N 1/16; C12N 15/70; C12N 15/81; C12Q 1/44; C12R 1/19; C12R 1/645

(52) U.S. Cl. CPC C12N 15/1055; C12N 1/165; C12N 15/1086; C12N 15/70; C12N 15/81; C12Q 1/44; C12R 2001/19; C12R 2001/645; G01N 2333/922

(57) ABSTRACT

A method for preparing interaction plasmid library and a method for testing interactions between cDNA libraries are provided. The cDNA libraries could be inserted into the first multiple cloning site and the second multiple cloning site of the dual expression vector for testing the interactions between the different cDNA libraries.

### CROSS-REFERENCE TO RELATED APPLICATIONS

This disclosure claims priority to Chinese Patent Application NO: 2024114663808, filed with China Intellectual Property Office on Oct. 18, 2024, the entire contents of which are incorporated herein by reference.

### SEQUENCE LISTING

The sequence listing xml file submitted herewith, named “Sequence_Listing.xml”, created on Dec. 11, 2024, and having a file size of 65,246 bytes, is incorporated by reference herein.

### TECHNICAL FIELD

This disclosure relates to the field of yeast two-hybrid technology. Specifically this disclosure relates A method for preparing interaction plasmid library and a method for testing interaction between cDNA libraries.

### BACKGROUND

cDNA library refers to the collection of all cDNA colonies formed by reverse transcription of mRNA transcribed at a certain developmental stage of an organism. cDNA plasmid library is a group of recombinant DNA colonies formed by connecting these cDNA colonies with appropriate vectors (commonly used phage or plasmid vectors) and transforming recipient bacteria. These cDNA plasmid libraries record all mRNA information of these tissues or cells. The cDNA plasmid cDNA library is tissue-specific or cell-specific because it reflects the gene encoding a protein expressed in a certain tissue or cell at a specific stage of development.

Protein to protein interaction (PPI) forms a major component of the biochemical reaction network within cells. Yeast two-hybrid system is widely used in the study of PPI. When the bait protein binds to the gene promoter of the prey protein and initiates the expression of the gene of the prey protein in yeast cells, if the gene expression of the prey protein is detected, it indicates that there is interaction between the prey protein and the bait protein; otherwise, there is no interaction between the two proteins. Micro-quantified or arrayed yeast two-hybrid systems are suitable for large-scale study of protein or cDNA interactions. The prior art also provides single-hybridization systems, three-hybridization systems, and reverse hybridization systems.

However, when the protein interaction is identified by the yeast two-hybrid system, at least two vectors, such as a capture vector and a bait vector, need to be constructed on the foreign genes under study, and the two vectors are transferred into two yeast strains respectively for expression and hybridization culture, so as to determine whether the two are hybridized. When it is necessary to test multiple pairs of proteins, it is necessary to construct multiple vectors accordingly. The traditional yeast two-hybrid system will greatly increase the workload of identification of multiple protein interactions or protein library interactions, and the efficiency of identification is severely limited.

### SUMMARY

One aspect, embodiments disclose a method for preparing an interaction plasmid library. The method includes: providing a dual expression vector with a first multiple cloning site and a second multiple cloning site; providing a first cDNA library and a second cDNA library; linearizing the dual expression vector; and respectively inserting the first cDNA library into the first multiple cloning site and the second cDNA library into the second multiple cloning site of the linearized dual expression vector. The dual expression vector also has a first sequence and a second sequence. The first sequence includes a orderly linked sequence consisting of a first multiple cloning site, a coding region of the GAL4 activation domain, a first promoter, a first operon, and a first initiator. The first promoter, the coding region of the GAL4 activation domain, and the first multiple cloning site have the same transcribed direction. The first promoter promotes the expression of the coding region of the GAL4 activation domain and the first cDNA library to be inserted at the first multiple cloning site. The second sequence includes a orderly linked sequence consisting of a second multiple cloning site, a coding region of the GAL4 binding domain, a second promoter, a second operon, and a second initiator. The second promoter, the coding region of the GAL4 binding domain, and the second multiple cloning site have the same transcribed direction. The second promoter promotes the expression of the coding region of the GAL4 binding domain and the second cDNA library to be inserted at the second multiple cloning site. The first sequence and the second sequence are joined to form a circular DNA molecule.

One aspect, embodiments disclose a method for testing interactions between cDNA libraries. The method includes: providing an interaction plasmid library provided by the above aspect, transforming the interaction plasmid library into yeast to obtain a recombinant yeast, and testing interactions between the first cDNA library and the second cDNA library on the growth state of the recombinant yeast on a selective culture medium.

The term “dual expression vector” refers to a vector capable of expressing two genes or two cDNA libraries simultaneously in yeast cells. The vector is used to test the interaction between these two genes or two cDNA libraries in yeast.

### BRIEF DESCRIPTION OF DRAWINGS

FIG. 1 shows the schematic diagram of a dual expression vector according to embodiments.

FIG. 2 shows the schematic diagram of a dual expression vector according to embodiments.

FIG. 3 shows the gel electrophoresis of AD library and BD library according to embodiments.

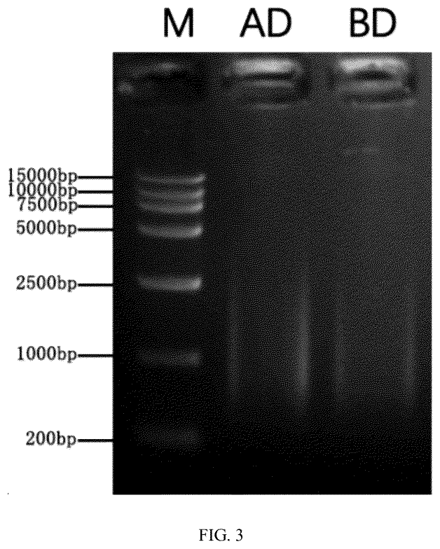

FIG. 4 shows the gel electrophoresis of the linearized pGAB-Dual Drive-IF-1 plasmid fragment according to embodiments.

FIG. 5 shows the gel electrophoresis of PCR for transformed colonies of the interaction plasmid library according to embodiments.

FIG. 6 shows the gel electrophoresis of the linearized pGAB-Dual Drive-IF-2 plasmid fragment according to embodiments.

FIG. 7 shows the plate image of transformants of the self-activating plasmid library according to embodiments.

FIG. 8 shows the growth status of recombinant transformants from the co-transformed plasmid library on 40 four-deficient plates after 3 days of incubation according to embodiments.

FIG. 9 shows the growth status of the recombinant transformants of the self-activating plasmid library on 8 quadruple dropout plates after 3 days according to embodiments.

FIG. 10 shows the gel electrophoresis of PCR amplification products from the co-transformed plasmid library or self-activating plasmid library according to embodiments.

### DETAILED DESCRIPTION OF THE EMBODIMENTS

In order to make the purpose, technical scheme and advantages of this application more clear, the following combined embodiments are further explained in detail. It will be understood that the specific embodiments described herein are intended only to explain the application and are not intended to qualify the application. The reagents not separately described in detail in this application are all routine reagents and can be obtained from commercial sources. The methods not specified in detail are routine experimental methods and can be known from the prior art.

### Prepare the Interaction Plasmid Library

Some embodiments provide a method for preparing an interaction plasmid library. The method includes: providing a dual expression vector with a first multiple cloning site and a second multiple cloning site; providing a first cDNA library and a second cDNA library; linearizing the dual expression vector; and respectively inserting the first cDNA library into the first multiple cloning site and the second cDNA library into the second multiple cloning site of the linearized dual expression vector. The dual expression vector also has a first sequence and a second sequence. The first sequence includes a orderly linked sequence consisting of a first multiple cloning site, a coding region of the GAL4 activation domain, a first promoter, a first operon, and a first initiator. The first promoter, the coding region of the GAL4 activation domain, and the first multiple cloning site have the same transcribed direction. The first promoter promotes the expression of the coding region of the GAL4 activation domain and the first cDNA library to be inserted at the first multiple cloning site. The second sequence includes a orderly linked sequence consisting of a second multiple cloning site, a coding region of the GAL4 binding domain, a second promoter, a second operon, and a second initiator. The second promoter, the coding region of the GAL4 binding domain, and the second multiple cloning site have the same transcribed direction. The second promoter promotes the expression of the coding region of the GAL4 binding domain and the second cDNA library to be inserted at the second multiple cloning site. The first sequence and the second sequence are joined to form a circular DNA molecule.

In some embodiments, the dual expression vector also has a terminator. The terminator is located between the transcription terminal of the first multiple cloning site and the transcription terminal of the second multiple cloning site. In some embodiments, the terminator is SEQ ID NO:11. The terminator terminates the expression of the coding region of the GAL4 activation domain in the first sequence and the expression of the first cDNA library inserted in the first multiple cloning site, and the expression of the GAL4 binding domain and the second cDNA library inserted in the second multiple cloning site, respectively.

In some embodiments, the first multiple cloning site has at least two restriction enzyme sites. The second multiple cloning site also has at least two restriction enzyme sites too.

In some embodiments, the dual expression vector also has one T7 promoters inserted between the first multiple cloning site and the coding region of the GAL4 activation domain, and another T7 promoter between the second multiple cloning site and the coding region of the GAL4 binding domain, respectively.

In some embodiments, both the first promoter and the second promoter are promoters suitable for yeast.

In some embodiments, the first promoter and the second promoter are independently selected from GK (phosphoglycerol kinase) promoter, GAP (glyceraldehyde 3-phosphate dehydrogenase) promoter, ADH (alcohol dehydrogenase) promoter, G3P (glyceraldehyde 3-phosphate dehydrogenase) promoter, ICL1 (isocitrate lyase) promoter, AOX1 (alcohol oxidase 1) promoter, TEF (transcription elongation factor EF-la) type promoters, GAL1 (galactokinase) type promoters, GAL1 (galactokinase gene) type promoters, Trp1 (tryptophan operon) promoters or promoters derived from them.

In some embodiments, the first promoter is an ADH1 promoter.

In some embodiments, the second promoter is a truncated ADH1 promoter.

In some embodiments, the first operon includes a first operator gene and a promoter for promoting the first operator gene. The second operon includes a second operator gene and a promoter for promoting the second operator gene.

In some embodiments, the first operator gene and the second operator gene are independently selected from LEU2, TRP1, or HIS. For example, the first operator gene is LEU gene, and its promoter is the LEU2 promoter. For example, the second operator gene is TRP gene, and its promoter is the TRP1 promoter.

In some embodiments, the second sequence also includes an third initiator located between the second promoter and the second gene operator, and a resistance gene connected to the transcriptional start end of the second initiator. The resistance gene has a transcription direction same to the third initiator. The third initiator has a transcription direction opposite to the GAL4 binding domain.

In some embodiments, the resistance gene is selected from one of Ampicillin, Tetracycline, Chloramphenicol, Streptomycin, Hygromycin, Spectinomycin, card Kanamycin, Blasticidin, Geneticin, Hygromycin B, Mycophenolic acid Acid, Puromycin, Zeocin, or Neomycin.

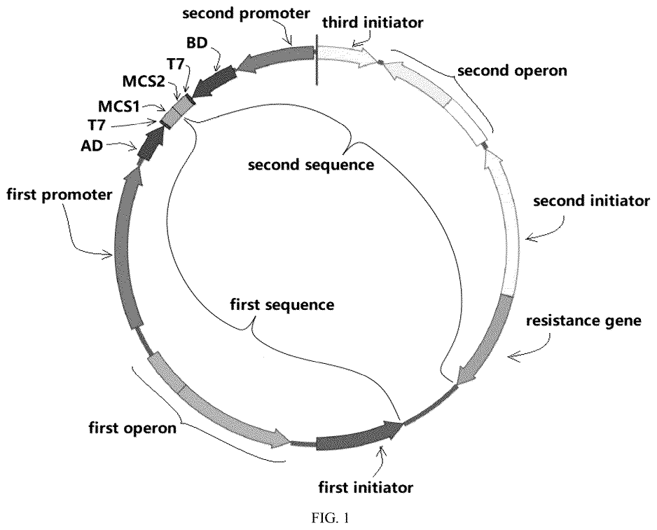

As shown in FIG. 1, the nucleotide sequence of the dual expression vector starts with a third initiator shown as SEQ ID NO.1. It is sequentially arranged through the second operon shown as SEQ ID NO:2, the second initiator shown as SEQ ID NO:3, the Kanamycin resistance gene shown as SEQ ID NO:4, the first initiator shown as SEQ ID NO:5, the first operon shown as SEQ ID NO:6, the first promoter shown as SEQ ID NO:7, the GAL4 activation domain shown as SEQ ID NO:8, the T7 promoter shown as SEQ ID NO:9, the MCS1 shown as SEQ ID NO:10, the terminator shown as SEQ ID NO:11, the MCS2 shown as SEQ ID NO:12, the GAL4 DNA binding domain shown as SEQ ID NO:13, and the second promoter shown as SEQ ID NO:14. And the nucleotide sequences shown in the sequence listing xml file are all forward strand sequences in this order.

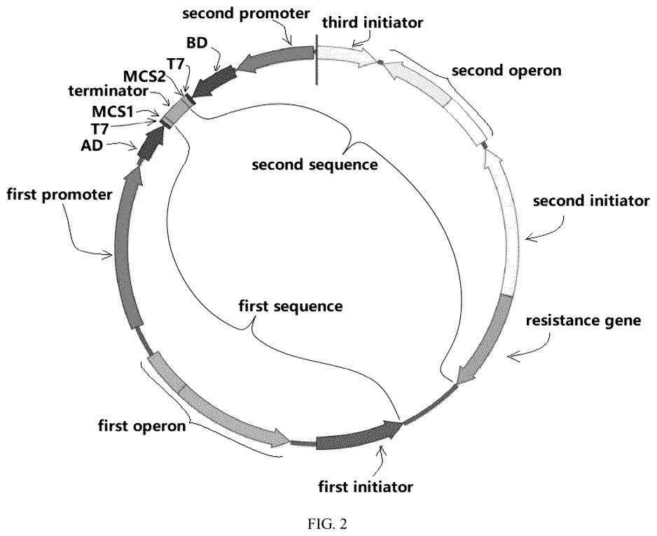

FIG. 2 shows the structure of the dual expression vector according to some embodiments. The first sequence includes a orderly linked sequence consisting of a first multiple cloning sites (MCS1, SEQ ID NO: 10), a T7 promoter (T7, SEQ ID NO: 9), a coding region of the GAL4 activation domain (AD, SEQ ID NO: 8), a first promoter (SEQ ID NO: 7), a first operon (SEQ ID NO: 6), and a first initiator (SEQ ID NO: 5).

The second sequence includes a orderly linked sequence consisting of a second multiple cloning sites (MCS2, SEQ ID NO: 12), a T7 promoter (T7, SEQ ID NO: 9), a coding region of the GAL4 DNA binding domain (BD, SEQ ID NO: 13), a second promoter (SEQ ID NO: 14), a third initiator (SEQ ID NO: 1), a second operon (SEQ ID NO: 2), a second initiator (SEQ ID NO: 3), and a kanamycin resistance gene (Kana, SEQ ID NO: 4).

As shown in FIG. 1 and FIG. 2, the transcription direction of each region on the dual expression vector is indicated by its arrow.

As shown in FIG. 1 and FIG. 2, the dual expression vector also has a sequence as SEQ ID NO:15 between the kanamycin resistance gene and the first initiator, and a sequence as SEQ ID NO:16 between the first operon and the first promoter.

### Prepare the Dual Expression Vector

Some embodiments provide a method for preparing the dual expression vector according to the above embodiments. The method includes: providing a basic vector consisting of the first sequence and the second sequence; synthesizing the terminator; and inserting the terminator between the transcription end of the first sequence and the transcription end of the second sequence to form a circular DNA molecule.

Other embodiments also provide a method for preparing the dual expression vector according to the above embodiments. The method includes: synthesizing a third fragment orderly including the first multiple cloning site, the T7 promoter, the coding region of the GAL4 activation domain and the first promoter; synthesizing a fourth fragment including the first operon; synthesizing a fifth fragment including the first initiator, the resistance gene and the second initiator; synthesizing a sixth fragment including the second initiator, the second operon, the third initiator, the second promoter, the coding region of the GAL4 binding domain, the T7 promoter and the second multiple cloning site; homologously recombining the third fragment, the fourth fragment, the fifth fragment and the sixth fragment; transferring the homologous recombination product into Escherichia coli; screening positive colonies from the transformants; extracting the basic vector from the culture of the positive colonies.

In some embodiments, the step of synthesizing the third fragment includes: amplifying a pGADT7 plasmid (Coolaber) by primers F3 (SEQ ID NO:17) and R3 (SEQ ID NO:18), and a high-fidelity enzyme; subjecting the amplification product to the gel electrophoresis; and recovering the third fragment (SEQ ID NO:19) from the gel electrophoresis.

In some embodiments, the step of synthesizing the fourth fragment includes: amplifying a pGADT7 plasmid by primers F4 (SEQ ID NO:20) and R4 (SEQ ID NO:21), and a high-fidelity enzyme; gel electrophoresing the amplification product; and recovering the fourth fragment (SEQ ID NO:22) from the gel electrophoresis.

In some embodiments, the step of synthesizing the fifth fragment includes: amplifying a pGBKT7 plasmid (Coolaber) by primers F5 (SEQ ID NO:23) and R5 (SEQ ID NO:24), and a high-fidelity enzyme; gel electrophoresing the amplification product; and recovering the fifth fragment (SEQ ID NO:25) from the gel electrophoresis.

In some embodiments, the step of synthesizing the sixth fragment includes: amplifying the pGBKT7 plasmid by primers F6 (SEQ ID NO:26) and R6 (SEQ ID NO:27), and a high-fidelity enzyme; gel electrophoresing the amplification product; and recovering the sixth fragment (SEQ ID NO:28) from the gel electrophoresis.

In these embodiments, PCR amplification is performed by using the 2×Phanta Max Master Mix High Fidelity Enzyme and ReAGAPent Kit from Vazyme, and the instructions for use can be found on the company's website at <https://www.vazyme.com/product/120.html>. The PCR products could be recovered and purified by using the FastPure Gel DNA Extraction Mini Kit from Vazyme, and the instructions for use could be found on the company's website at <https://www.vazyme.com/companyfile/2149.html>.

The steps also include: mixing 40 ng of the third fragment, 36 ng of the fourth fragment, 70 ng of the fifth fragment, 66 ng of the sixth fragment, and 10 μL of 2×CE Mix (from Vazyme ClonExpress Ltra One Step Cloning Kit V2) in a total volume of 20 μL; performing the mixture at 50° C. for 30 minutes; cooling down to 4° C. or immediately placing on ice for cooling; transforming the recombinant into Escherichia coli to obtain positive clones; cultivating the positive colonies; extracting the basic vector from the culture of the positive colonies.

### Prepare the Interaction Plasmid Library

In some embodiments, the first cDNA library (used as the AD library) and the second cDNA library (used as the BD library) are simultaneously inserted into the dual expression vector provided in the example, and a interaction plasmid library carrying both the first cDNA and the second cDNA is prepared. The specific steps are as follows.

### 1. Extract Total RNA

Total RNA was extracted from maize SAM tissue using the Trizol method.

### 2. Synthesize the AD Library and BD Library

The total RNA were reversely transcribed to obtain the cDNA strands. The cDNA strands were amplified by PCR to obtain the cDNA library.

In some steps, 0.2-4.0 μg purified total RNA, 1.0 μL CDS primer, 1.0 μL dNTP, and the rest of deionized water were mixed to form a 7.5 μL solution. The 7.5 μL solution was incubated at 65° C. for 5 minutes and then placed on ice for 2 to 3 minutes to obtain the sample solution for reverse transcription. Then, 7.88 μL of the sample solution, 1.0 μL of SuperScript IV reverse transcriptase (2000 U/μL), 1.0 μL of RNase inhibitor (40 U/μL), 4.0 μL of SuperScript IV first-strand buffer (5×), 1 μL of DTT (100 mM), 4 μL of betaine (5M), 0.12 μL of MgCl₂ (1M), and 1 μL of TSO primer (1 μM) were mixed to form the reverse transcription mixture. The reverse transcription mixture was incubated at 55° C. for 10 minutes and 80° C. for 10 minutes to obtain the cDNA strand.

In some embodiments, a PCR reaction was carried out in a 50 μL mixture consisting of 1.0-5.0 μL of the cDNA strand, 1.0 μL of AD-F, 1.0 μL of AD-R, 25 μL of KAPA HiFi HotStart High Fidelity Enzyme Premix (Roche, KK2602), and the remaining deionized water. The PCR products were detected by electrophoresis. The optimal number of PCR amplification cycles was selected according to the brightness and size distribution of the bands, and then PCR amplification was performed to obtain 200 μL of cDNA library products. The PCR reaction program included: one cycle of reaction at 95° C. for 3 minutes; 11, 13, 15, 17 or 19 cycles of reaction at 98° C. for 20 seconds, at 62-67° C. for 15 seconds and at 72° C. for 3 minutes, and the annealing temperature was determined according to the primers. The reaction lasted for one cycle at 72° C. for 5 minutes; and one cycle at 4° C. for 1 minute.

Table 1 shows the primers. Among them, “r” refers to ribonucleotide, “N” stands for A, C, G or T, and “V” refers to A, G or C. “AD” refers to the primers for synthesizing the AD library, and “BD” refers to the primers for synthesizing the BD library.

TABLE 1
Primers

| Primer name | Sequence (5′→3′) |
|---|---|
| CDS primer | ggtcgccgtatcattacgactcatTTTT |
| (AD) | TTTTTTTTTTTTTTTTTTTTTTTTTTVN, |
| | SEQ ID NO: 29 |
| CDS primer | gccgtcttctgcttgtctacgattcatT |
| (BD) | TTTTTTTTTTTTTTTTTTTTTTTTTTTT |
| | TVN, |
| | SEQ ID NO: 30 |
| TSO(AD) | gtaccagattacgct/rG//rG//rG/, |
| | SEQ ID NO: 31 |
| TSO(BD) | tcagaggaggacctg/rG//rG//rG/, |
| | SEQ ID NO: 32 |
| AD-F | tacgacgtaccagattacgct, |
| | SEQ ID NO: 33 |
| AD-R | tcggtggtcgccgtatcatt, |
| | SEQ ID NO: 34 |
| BD-F | tcggtggtcgccgtatcatt, |
| | SEQ ID NO: 35 |
| BD-R | cgtatgccgtcttctgcttg, |
| | SEQ ID NO: 36 |

In some embodiments, the obtained cDNA library products were purified using KAPA Pure Beads (Roche, KK8000) and sorted in three proportional ranges of 0.4 (>2000 bp), 0.4-0.5 (2000 bp−1000 bp), and 0.5-0.6 times the product volume. AD library and BD library were obtained respectively, and tested by electrophoresis.

FIG. 2 shows the visible dispersion bands. This suggests that the obtained AD library and BD library fragments have a high degree of uniformity.

### 3. Prepare the Interaction Plasmid Library

Examples had provided the steps for constructing an interaction plasmid library. The steps included: linearizing a dual expression vector (shown as the FIG. 1); recombining the linearized dual expression vector, the sequence shown in SEQ ID NO: 11, the AD library and the BD library; transforming the recombinant into Escherichia coli to obtain positive colonies; cultivating the positive colonies; extracting the interaction plasmid library from the culture of the positive colonies.

Examples had provided the steps for constructing an interaction plasmid library. The steps included: linearizing a dual expression vector (shown as the FIG. 2); recombining the linearized dual expression vector, the AD library and the BD library; transforming the recombinant into Escherichia coli to obtain positive colonies; cultivating the positive colonies; extracting the interaction plasmid library from the culture of the positive colonies.

Among them, the step of linearizing a dual expression vector included: transforming the dual expression vector (shown as the FIG. 1) into Escherichia coli; cultivating the transformants at 37° C. for one day; picking the colonies to culture in LB medium containing kanamycin antibiotics for 12 to 16 hours; and extracting the dual expression vectors (concentration >100 ng/μL) with the Plasmid DNA Mini Kit 1 (Omega Bio-tek); and digesting the dual expression vectors with BamH I-HF and NdeI to obtain the linearized dual expression vector. The digestion system was prepared with 5 μg yeast dual expression vector, 5 μL BamH I-HF, 5 μL NdeI, 10 μL 10× cutsmart buffer and 85 μL deionized water, and incubated at 30° C. for 4 h for double enzyme digestion. The digestion products were detected by electrophoresis, and purified by Cycle Pure Kit (Omega Bio-tek) to obtain linear yeast dual expression vector.

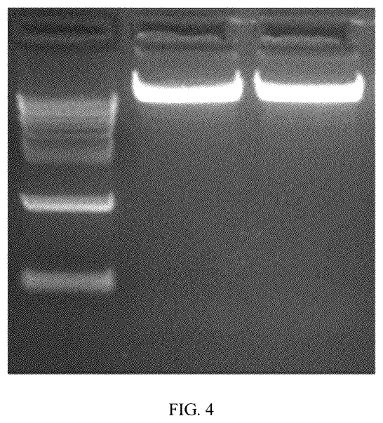

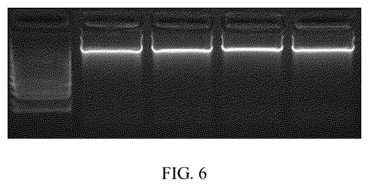

FIG. 4 shows a single band of dual expression vector (shown as the FIG. 1) on the picture of electrophoresis detection. FIG. 6 shows a single band of dual expression vector (shown as the FIG. 2) on the picture of electrophoresis detection.

Among them, the recombination reaction system included 30 to 60 μg (1 μL) of the AD library, 30 to 60 μg (1 μL) of the BD library product, 10 μg (1 μL) of the sequence shown in SEQ ID NO:11 sequence, 200 g (1 to 2 μL) of the linearized dual expression vector(shown as the FIG. 1), 5 μL of 2×CE Mix and residual deionized water in a total volume of 10 μL. The reaction performed at 50° C. for 30 min, cooled down to 4° C.

Among them, the recombination reaction system included 30 to 60 μg (1 μL) of the AD library, 30 to 60 μg (1 μL) of the BD library product, 200 μg (1 to 2 μL) of the linearized dual expression vector(shown as the FIG. 2), 5 μL of 2×CE Mix and residual deionized water in a total volume of 10 μL. The reaction performed at 50° C. for 30 min, cooled down to 4° C.

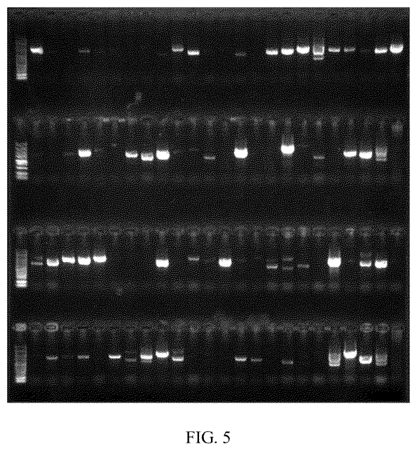

Each plasmid in the interacting plasmid library obtained by the embodiment carries two cDNA fragments, one for the AD acting element and another for the BD acting element, for interaction detection in yeast. As shown in FIG. 5, the obtained transformants are spread on 30 plates, and 96 single colonies are randomly picked. Among them, 72 single colonies are able to amplify bands of different sizes. The results preliminarily indicates each plasmid in this interaction plasmid library simultaneously carries both AD sequences and BD sequences.

In some contrastive examples, the dual expression vector (shown as the FIG. 2) was linearized with BspdI and SacI. A recombination reaction system with a total volume of 10 μL was prepared by mixing 200 μg (1-2 μL) of the linearized dual expression vector (shown as the FIG. 2), 60 μg (1 μL) of the BD library products, 5 μL of 2×CE Mix and the remaining deionized water. The recombinant was transformed into Escherichia coli in the same way as the above examples, and positive colonies were screened, and the self-activated plasmid library was extracted from the cultures of the positive colonies. Each plasmid in the obtained self-activated plasmid library had carried one cDNA fragment using as the BD acting element. The self-activated plasmid libraries were used to compare with the interaction plasmid libraries described above to discover self-activated genes in cDNA libraries.

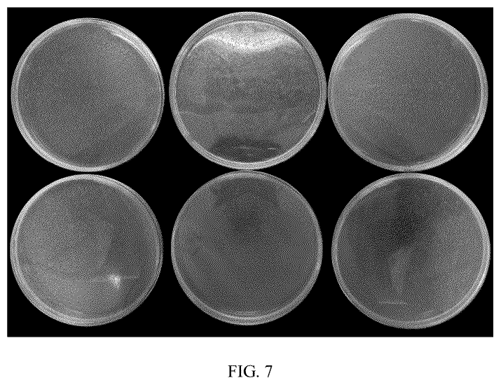

FIG. 7 shows the growth status of the self-activated transformants after being cultured on 6 plates for 16 hours.

### 4. Test the Interaction Plasmid Library Interactions

Competent yeasts Y2HGold were prepared. The interaction plasmid library and the self-activated plasmid library obtained from the above examples were transformed into the competent Y2HGolds respectively. The transformants were spreaded on the medium SD/-Leu/-Trp/-Ade/-His. The positive colonies were detected by colony PCRs. The interaction plasmid library and self-activated plasmid library were extracted from the positive colonies, respectively, and amplified by PCR, and sequenced the amplified products of PCR.

In some embodiments, the steps for preparing competent yeast Y2HGol included:
1) The yeast Y2HGold strain (Weidi Biotechnology: CAT #: YC1002) was streaked and cultured on the YPDA solid plate (Coolaber, PM2011−500 μg+agar, CA1331−500 μg). Then, the fresh single colonies were picked and transferred into 5 mL of YPDA liquid medium (in a 50 mL centrifuge tube). The culture was shaken at 30° C. and 250 rpm for 12 hours. After that, 50 μL of the bacterial suspension was inoculated into 50 mL of YPDA liquid medium (in a 250 mL Erlenmeyer flask) and shaken at 250 rpm until the OD600 was approximately 0.15-0.3 (for 16-20 hours).
2) The yeast suspension was collected and centrifuged at 700 μg for 5 minutes to collect the cells. The supernatant was discarded, and the cells were resuspended in 100 mL of YPDA liquid medium. The culture was then shaken at 30° C. and 250 rpm until the OD600 reached 0.4-0.5.
3) The yeast suspension was collected and centrifuged at 700 μg for 5 minutes to collect the cells. The supernatant was discarded, and the cells were resuspended in 50 mL of sterile ddH2O.
4) The yeast suspension was collected and centrifuged at 700 μg for 5 minutes to collect the cells. The supernatant was discarded, and the cells were resuspended in 15 mL of 1.1×TE/liAC (Weidi Biotechnology: YC4210 μL) pre-cooled at 4° C.
5) The yeast suspension was collected and centrifuged at 1500 μg for 30 seconds. The supernatant was discarded, and the cells were resuspended in 0.6 mL of 1.1×TE/liAC. This is the solution of the competent yeast Y2HGold.

In some embodiments, the steps for transforming the interaction plasmid library and the self-activated plasmid library obtained from the above embodiments into this competent Y2HGold include:
1) The phosphate buffer solution with a pH of 7.0 containing 10 mg/mL salmon extract DNA (Kermel) was placed in a water bath at 95° C. for 3 minutes and then in an ice bath for 3 minutes.
2) The interacting plasmid library or self-activated plasmid library obtained above was taken 5-15 μg, mixed with 40 μL salmon extract DNA solution, 600 μL yeast receptive state and 2.5 mL PEG/LiAc (YC5001M). The obtained mixture was placed in a water bath at 30° C. for 45 minutes, and inverted 6-8 times every 15 min to mix evenly.
3) 160 μL of sterile DMSO was added into the obtained mixture (Weidi Biotechnology: YC6020S).
4) The obtained mixture was placed in a water bath at 42° C. for 20 minutes, and inverted it 6-8 times every 10 minutes to mix evenly.
5) The obtained mixture was centrifuged at 4000 rpm for 40 seconds, and the supernatant was discarded. The collected yeast were resuspended with 2×YPDA or YPD Plus (Weidi Biotechnology: YC6006S) into 3 mL.
6) The obtained mixture was shaken at 30° C. and 200 rpm for 1.5 hours, centrifuged for 30 seconds, and the supernatant was discarded.
7) The collected yeast were resuspended with 18 mL 0.9% NaCl. 300 μL suspended yeast solution was spread and cultured at 30° C. for 48-72 h.
8) 300 μL yeast solution, 300 μL of the 10-fold diluted yeast suspension (diluted with double distilled water) and 300 μL of the 100-fold diluted yeast suspension were spread on the double dropout plates SD/-Leu-Trp respectively to test the transformation efficiency. In addition, 300 μL of the transformant yeast suspension of the interaction plasmid library and 300 μL of the transformant yeast suspension of the self-activated plasmid library were spread on the quadruple dropout plates SD/-Leu/-Trp/-Ade/-His to to screen interacting genes in cDNA library and self-activating genes in cDNA library.

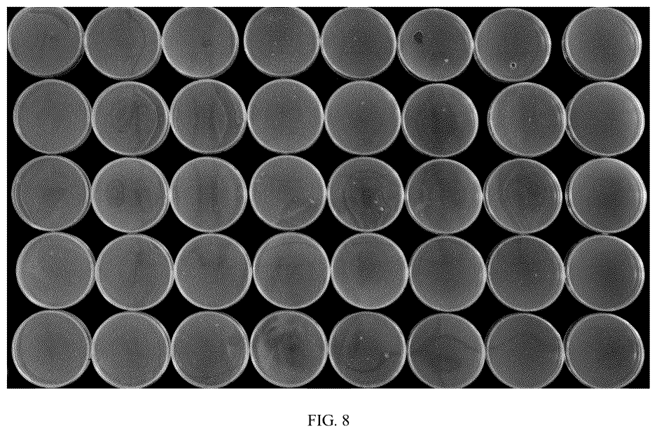

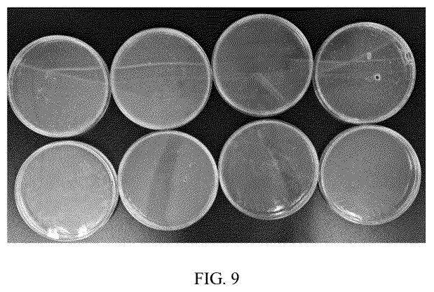

FIG. 8 shows the growth status of the recombinants of the interaction plasmid library on 40 quadruple dropout plates after 3 days. FIG. 9 shows the growth status of the recombinant of the self-activated plasmid library on 8 quadruple dropout plates after 3 days.

In some embodiments, the steps for detecting the positive colonies included:
1) The lysate of the transformants obtained from the above embodiments were prepared.
2) The lysate was thermatively denatured at 80° C. for 15 min, centrifuged at low speed, and the supernatant templates of 1˜-5 μL were taken, and CX-Primer-F (SEQ ID NO:37) and CX-Primer-R (SEQ ID NO:38) were used as primers for PCR. PCR products were electrophoretically detected using 1% agarose gel, and positive colonies were identified based on electrophoretic bands.

In some embodiments, the steps for extracting cDNA plasmids and self-activated plasmids from positive colonies include:
1) Positive colonies were collected from the quadruple dropout interaction screening plates cultured for 2 to 4 days or the self-activated plates cultured for 6 to 7 days. The obtained yeast liquid was centrifuged at 12,000 rpm for 1 minute at room temperature. The supernatant was discarded and the precipitate was the collected yeast cells.
2) The collected yeast cells were resuspended in the lytic enzyme solution dissolved at 37° C. (Classic Yeast Plasmid Mini-Prep Kit (including lyticase), PE054, Coolaber; this kit includes Package A and Package B. Package A is the lytic enzyme solution. Package B includes Solution I, Solution II, Solution III, Solution IV, and the elution buffer matrix plasmid adsorption column). After incubating in a shaker at 37° C. (200-250 rpm) for 1 to 2 hours, the obtained lytic enzyme solution was centrifuged at 12,000 rpm for 1 minute at room temperature. The supernatant was discarded and the precipitate obtained was the lysed yeast cells.
3) Solution II and Solution III were preheated in a 40° C. water bath until the precipitate was completely dissolved and then left to stand at room temperature for later use.
4) 180 μL of Solution I was added to the yeast cell precipitate, and the mixture was thoroughly mixed by pipetting or vortexing.
5) 180 μL of Solution II was added, and the tube was inverted 6-8 times to mix thoroughly. Then it was left to stand at room temperature for 5 minutes.
6) 440 μL of Solution III was added, and the tube was immediately inverted to mix thoroughly.
7) The mixture was centrifuged at 12,000 rpm for 10 minutes at room temperature. The supernatant was carefully aspirated into the adsorption column, left to stand at room temperature for 2 minutes, and then centrifuged at 12,000 rpm for 1 minute at room temperature. The flow-through was discarded. Note: There may be white suspended substances after centrifugation. Just avoid them when aspirating the supernatant. A small amount of suspended substances will not affect the extraction result.
8) 500 μL of Washing Solution IV was added to the adsorption column, and the column was centrifuged at 12,000 rpm for 1 minute at room temperature. The flow-through was discarded.
9) The adsorption column was placed back into the collection tube and centrifuged at 12,000 rpm for 2 minutes at room temperature.
10) The adsorption column was transferred to a new 1.5 mL centrifuge tube. 30-50 μL of the elution buffer (preheated to 65-70° C.) was added to the center of the adsorption column. It was left to stand at room temperature for 2 minutes and then centrifuged at 12,000 rpm for 1 minute at room temperature.
11) The flow-through was the obtained interaction plasmid library or self-activated plasmid library. PCR was performed on it using detection steps such as colony PCR. The amplified products were detected by electrophoresis. If there was a single bright band without smearing, it indicated that the extracted interaction plasmid library or self-activated plasmid library was intact without degradation.

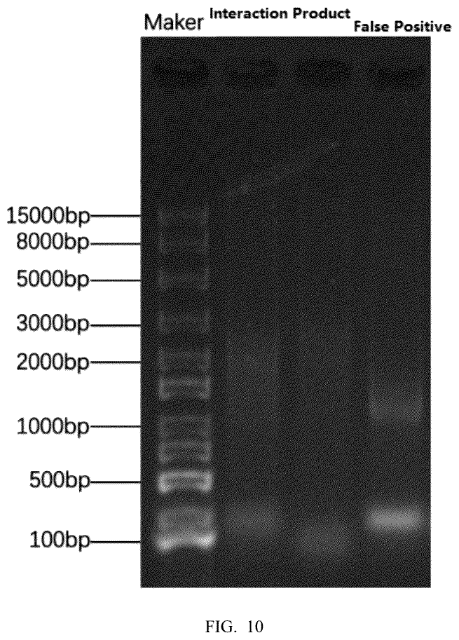

The specific steps of amplifying and sequencing the interaction plasmid library and the self-activated plasmid library included:
1) The interaction plasmid library or the self-activated plasmid library was prepared into an amplification system as shown in Table 2, and PCR amplification was carried out according to Table 3. The products of the interaction plasmid library and the interaction plasmid library could be sorted by adding different combinations of barcode. For example, different barcode combination types could be added during amplification, and after being mixed and sequenced (third-generation sequencing), the sorting analysis could finally be done through the sequencing data. For example, 5 μgroups of primers containing barcodes were provided in the embodiments to generate 25 combination types. Among them, AD and BD were used in combination. For example, AD-barcode1+BD-barcode2 was used for amplifying the products of the interaction experiment; AD-barcode1+BD-barcode3 was used for amplifying the self-activated products. As shown in FIG. 10, the quality of the amplified products is relatively good.

AD-barcode1:
SEQ ID NO: 39
GCGCTCTGTGTGCAGCcatacgacgtaccagattacg,
AD-barcode2:
SEQ ID NO: 40
TCATGAGTCGACACTAcatacgacgtaccagattacg,
AD-barcode3:
SEQ ID NO: 41
TATCTATCGTATACGCcatacgacgtaccagattacg,
AD-barcode4:
SEQ ID NO: 42
ATCACACTGCATCTGAcatacgacgtaccagattacg,
AD-barcode5:
SEQ ID NO: 43
ACGTACGCTCGTCATAcatacgacgtaccagattacg,
BD-barcode1:
SEQ ID NO: 44
GCGCTCTGTGTGCAGCcatcatggaggagcagaagct,
BD-barcode2:
SEQ ID NO: 45
TCATGAGTCGACACTAcatcatggaggagcagaagct,
BD-barcode3:
SEQ ID NO: 46
TATCTATCGTATACGCcatcatggaggagcagaagct,
BD-barcode4:
SEQ ID NO: 47
ATCACACTGCATCTGAcatcatggaggagcagaagct,
BD-barcode5:
SEQ ID NO: 48
ACGTACGCTCGTCATAcatcatggaggagcagaagct,

2) 5 μL products were respectively taken out from the 13th, 15th, and 17th cycles of PCR amplification and loaded together with 0.1 μg of 5-kb DNA size marker into a 1.5% agarose Golden view gel in 1× TAE buffer for determining the optimal cycle number. The amplified products were purified with the KAPA Pure Beads kit, and the 500-bp purified products were sequenced.

TABLE 2
PCR Reaction System

| Ingredient | Amount |
|---|---|
| Interaction plasmid library or | 5.0 μL (150 ng) |
| Self-activated plasmid library | |
| Deionized H₂O | 18 μL |
| AD-barcode-N | 1.0 μL |
| BD-barcode-N | 1.0 μL |
| KAPA HiFi HotStart ReadyMix | 25 μL |
| Total | 50 μL |

TABLE 3
PCR Reaction Procedure

| Cycle number | procedure |
|---|---|
| 1 | 95° C. 3 min |
| 18/20/22 | 98° C. 20 s; 67° C. 15 s; 72° C. 5 min |
| 1 | 72° C. 5 min |
| 1 | 4° C. 1 min |

As mentioned above, these are only the preferred specific implementation methods of this application. However, the protection scope of this application is not limited to this. Any person skilled in the art can easily think of changes or substitutions within the technical scope disclosed in this application, which should be covered within the protection scope of this application.

What is claimed is:

1. A method for preparing an interaction plasmid library comprising:
providing a dual expression vector with a first multiple cloning site and a second multiple cloning site;
providing a first cDNA library and a second cDNA library;
linearizing said dual expression vector; and
respectively inserting said first cDNA library into said first multiple cloning site and said second cDNA library into said second multiple cloning site of the linearized dual expression vector;
said dual expression vector comprising:
a first sequence, said first sequence comprises a orderly linked sequence consisting of a first multiple cloning site, a coding region of the GAL4 activation domain, a first promoter, a first operon, and a first initiator, said first promoter, said coding region of the GAL4 activation domain, and said first multiple cloning site have the same transcribed direction, said first promoter promotes the expression of said coding region of the GAL4 activation domain and said first cDNA library to be inserted at said first multiple cloning site; and
a second sequence, said second sequence comprises a orderly linked sequence consisting of a second multiple cloning site, a coding region of the GAL4 binding domain, a second promoter, a second operon, and a second initiator, said second promoter, said coding region of the GAL4 binding domain, and said second multiple cloning site have the same transcribed direction, said second promoter promotes the expression of the coding region of the GAL4 binding domain and said second cDNA library to be inserted at the second multiple cloning site;
said first sequence and said second sequence are joined to form a circular DNA molecule.

2. The method according to claim 1, wherein said dual expression vector further comprises a terminator, said terminator is located between the transcription terminal of the first multiple cloning site and the transcription terminal of the second multiple cloning site.

3. The method according to claim 2, wherein said terminatior is SEQ ID NO:11.

4. The method according to claim 1, wherein said first multiple cloning site comprises at least two restriction enzyme sites.

5. The method according to claim 1, wherein said second multiple cloning site comprises at least two restriction enzyme sites.

6. The method according to claim 1, wherein said dual expression vector also comprises one T7 promoter between said first multiple cloning site and said coding region of the GAL4 activation domain, and another T7 promoter between said second multiple cloning site and said coding region of the GAL4 binding domain, respectively.

7. The method according to claim 1, wherein said coding region of the GAL4 activation domain is SEQ ID NO:8, said coding region of the GAL4 binding domain is SEQ ID NO:13.

8. The method according to claim 1, wherein said first promoter is elected from phosphoglycerol kinase promoter, glyceraldehyde 3-phosphate dehydrogenase promoter, alcohol dehydrogenase promoter, glyceraldehyde 3-phosphate dehydrogenase promoter, isocitrate lyase promoter, alcohol oxidase 1 promoter, transcription elongation factor EF-la type promoters, galactokinase type promoters, galactokinase gene type promoters, or tryptophan operon promoter;
said second promoter is elected from phosphoglycerol kinase promoter, glyceraldehyde 3-phosphate dehydrogenase promoter, alcohol dehydrogenase promoter, glyceraldehyde 3-phosphate dehydrogenase promoter, isocitrate lyase promoter, alcohol oxidase 1 promoter, transcription elongation factor EF-la type promoters, galactokinase type promoters, galactokinase gene type promoters, or tryptophan operon promoter.

9. The method according to claim 1, wherein said first promoter is SEQ ID NO:7, said second promoter is SEQ ID NO:14.

10. The method according to claim 1, wherein said first operon comprises a first operator gene and a promoter for promoting said first operator gene, said second operon comprises a second operator gene and a promoter for promoting said second operator gene.

11. The method according to claim 1, wherein said first operator gene is selected from LEU2, TRP1, or HIS, said second operator gene is selected from LEU2, TRP1, or HIS.

12. The method according to claim 1, wherein said first initiator is SEQ ID NO:5, said second initiator is SEQ ID NO:3.

13. The method according to claim 1, wherein said second sequence furthe comprising:
a resistance gene connecting to the transcriptional start end of said second initiator; and
an third initiator locating between said second promoter and said second gene operator, said third initiator is SEQ ID NO:1.

14. The method according to claim 13, wherein said resistance gene is selected from one resistance coding gene of Ampicillin, Tetracycline, Chloramphenicol, Streptomycin, Hygromycin, Spectinomycin, Kanamycin, Blasticidin, Geneticin, Hygromycin B, Mycophenolic Acid, Puromycin, Zeocin or Neomycin.

15. The method according to claim 1, wherein the step of linearizing said dual expression vector comprising:
digesting said dual expression vector with BamH I-HF and NdeI.

16. The method according to claim 1, wherein the step of respectively inserting said first cDNA library into said first multiple cloning site and said second cDNA library into second multiple cloning site of the linearized dual expression vector comprising:
recombining said first cDNA library, said second cDNA library, and the linearized dual expression vector;
transforming the recombinant into Escherichia coli;
screening the positive colonies from the transformant;
extracting said interaction plasmid libraries from the positive colonies.

17. The method according to claim 1, wherein the step of respectively inserting said first cDNA library into said first multiple cloning site and said second cDNA library into second multiple cloning site of the linearized dual expression vector comprising:
recombining said first cDNA library, said second cDNA library, the nucleic acid of SEQ ID NO:11 and the linearized dual expression vector;
transforming the recombinant into Escherichia coli;
screening the positive colonies from the transformant;
extracting said interaction plasmid libraries from the positive colonies.

18. A method for testing interactions between cDNA libraries comprising:
providing an interaction plasmid library;
transforming said interaction plasmid library into yeast to obtain a recombinant yeast; and
testing interactions between the first cDNA library and the second cDNA library with the growth state of the recombinant yeast on a selective culture medium.

19. The method according to claim 1, wherein said yeast is Y2H yeast.

20. The method according to claim 1, wherein said selective culture medium is selected from SD/-Leu/-Trp or SD/-Leu/-Trp/-His/-Ade.


SEQUENCE LISTING (48 sequences)

SEQ ID NO: 1 - DNA, 548 bp, synthetic construct
  source 1..548: mol_type = other DNA
atcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcat

SEQ ID NO: 2 - DNA, 1147 bp, synthetic construct
  source 1..1147: mol_type = other DNA
cctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgttgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcg

SEQ ID NO: 3 - DNA, 1346 bp, synthetic construct
  source 1..1346: mol_type = other DNA
gtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgt

SEQ ID NO: 4 - DNA, 923 bp, synthetic construct
  source 1..923: mol_type = other DNA
ggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctg

SEQ ID NO: 5 - DNA, 799 bp, synthetic construct
  source 1..799: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcc

SEQ ID NO: 6 - DNA, 1498 bp, synthetic construct
  source 1..1498: mol_type = other DNA
agcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtgggacacctgtagcttcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagtt

SEQ ID NO: 7 - RNA, 1472 nt, synthetic construct
  source 1..1472: mol_type = other RNA
cctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactcca

SEQ ID NO: 8 - DNA, 342 bp, synthetic construct
  source 1..342: mol_type = other DNA
gccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagag

SEQ ID NO: 9 - DNA, 23 bp, synthetic construct
  source 1..23: mol_type = other DNA
taatacgactcactatagggcga

SEQ ID NO: 10 - DNA, 27 bp, synthetic construct
  source 1..27: mol_type = other DNA
catatggccatggaggccagtggatcc

SEQ ID NO: 11 - DNA, 145 bp, synthetic construct
  source 1..145: mol_type = other DNA
aatgatacggcgaccaccgatatataaactcatttacttatgtaggaataaagagtatcatctttcaaagttgaaacgcgtcgctattttttagatgatactctttatttctagacagttatatacgtatgccgtcttctgcttg

SEQ ID NO: 12 - DNA, 28 bp, synthetic construct
  source 1..28: mol_type = other DNA
gagctcgagctgcagatgaatcgatacc

SEQ ID NO: 13 - DNA, 441 bp, synthetic construct
  source 1..441: mol_type = other DNA
cgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcat

SEQ ID NO: 14 - DNA, 707 bp, synthetic construct
  source 1..707: mol_type = other DNA
ggagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggat

SEQ ID NO: 15 - DNA, 585 bp, synthetic construct
  source 1..585: mol_type = other DNA
agcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 16 - DNA, 281 bp, synthetic construct
  source 1..281: mol_type = other DNA
gggggatctcgactctagctagaggatcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgataacttcgtataatgtatgctatacgaagttattaggtctgaagaggagtttacgtccagccaagctagc

SEQ ID NO: 17 - DNA, 22 bp, synthetic construct
  source 1..22: mol_type = other DNA
cctgcaggtcgagatccgggat

SEQ ID NO: 18 - DNA, 22 bp, synthetic construct
  source 1..22: mol_type = other DNA
cctgcaggtcgagatccgggat

SEQ ID NO: 19 - DNA, 2016 bp, synthetic construct
  source 1..2016: mol_type = other DNA
cctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggataaagcggaattaattcccgagcctccaaaaaagaagagaaaggtcgaattgggtaccgccgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagatctttaatacgactcactatagggcgagcgccgccatggagtacccatacgacgtaccagattacgctcatatggccatggaggccagtgaattccacccgggtgggcatcgatacgggatcc

SEQ ID NO: 20 - DNA, 35 bp, synthetic construct
  source 1..35: mol_type = other DNA
gaccatgattacgccttaagcaaggattttcttaa

SEQ ID NO: 21 - DNA, 30 bp, synthetic construct
  source 1..30: mol_type = other DNA
atctcgacctgcagggctagcttggctgga

SEQ ID NO: 22 - DNA, 1782 bp, synthetic construct
  source 1..1782: mol_type = other DNA
ttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtgggacacctgtagcttcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttgggggatctcgactctagctagaggatcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgataacttcgtataatgtatgctatacgaagttattaggtctgaagaggagtttacgtccagccaagctagc

SEQ ID NO: 23 - DNA, 20 bp, synthetic construct
  source 1..20: mol_type = other DNA
ggcgtaatcatggtcatagc

SEQ ID NO: 24 - DNA, 30 bp, synthetic construct
  source 1..30: mol_type = other DNA
cgcagatataggtgcgacgtgaacagtgag

SEQ ID NO: 25 - DNA, 3620 bp, synthetic construct
  source 1..3620: mol_type = other DNA
gacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatgacgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 26 - DNA, 21 bp, synthetic construct
  source 1..21: mol_type = other DNA
gcaacacgcagatataggtgc

SEQ ID NO: 27 - DNA, 21 bp, synthetic construct
  source 1..21: mol_type = other DNA
ctgatctcagaggaggacctg

SEQ ID NO: 28 - DNA, 3354 bp, synthetic construct
  source 1..3354: mol_type = other DNA
caggtcctcctctgagatcagcttctgctcctccatgatggcggctcgccctatagtgagtcgtattacaaattccggcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggcttgcttcaagcttggagttgattgtatgcttggtatagcttgaaatattgtgcagaaaaagaaacaaggaagaaagggaacgagaacaatgacgaggaaacaaaagattaataattgcaggtctatttatacttgatagcaagacagcaaacttttttttatttcaaattcaagtaactggaaggaaggccgtataccgttgctcattagagagtagtgtgcgtgaatgaaggaaggaaaaagtttcgtgtgcttcgagatacccctcatcagctctggaacaacgacatctgttggtgctgtctttgtcgttaattttttcctttagtgtcttccatcatttttttgtcattgcggatatggtgagacaacaacgggggagagagaaaagaaaaaaaaagaaaagaagttgcatgcgcctattattacttcaatagatggcaaatggaaaaagggtagtgaaacttcgatatgatgatggctatcaagtctagggctacagtattagttcgttatgtaccaccatcaatgaggcagtgtaattggtgtagtcttgtttagcccattatgtcttgtctggtatctgttctattgtatatctcccctccgccacctacatgttagggagaccaacgaaggtattataggaatcccgatgtatgggtttggttgccagaaaagaggaagtccatattgtacacccggaaacaacaaaaggatctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgttgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgc

SEQ ID NO: 29 - DNA, 56 bp, synthetic construct
  source 1..56: mol_type = other DNA
ggtcgccgtatcattacgactcatttttttttttttttttttttttttttttttvn

SEQ ID NO: 30 - DNA, 59 bp, synthetic construct
  source 1..59: mol_type = other DNA
gccgtcttctgcttgtctacgattcatttttttttttttttttttttttttttttttvn

SEQ ID NO: 31 - DNA, 18 bp, synthetic construct
  source 1..18: mol_type = other DNA
  misc_feature 16..18: note = RNA
gtaccagattacgctggg

SEQ ID NO: 32 - DNA, 18 bp, synthetic construct
  source 1..18: mol_type = other DNA
  misc_feature 16..18: note = RNA
tcagaggaggacctgggg

SEQ ID NO: 33 - DNA, 21 bp, synthetic construct
  source 1..21: mol_type = other DNA
tacgacgtaccagattacgct

SEQ ID NO: 34 - DNA, 20 bp, synthetic construct
  source 1..20: mol_type = other DNA
tcggtggtcgccgtatcatt

SEQ ID NO: 35 - DNA, 20 bp, synthetic construct
  source 1..20: mol_type = other DNA
tcggtggtcgccgtatcatt

SEQ ID NO: 36 - DNA, 20 bp, synthetic construct
  source 1..20: mol_type = other DNA
cgtatgccgtcttctgcttg

SEQ ID NO: 37 - DNA, 21 bp, synthetic construct
  source 1..21: mol_type = other DNA
catacgacgtaccagattacg

SEQ ID NO: 38 - DNA, 21 bp, synthetic construct
  source 1..21: mol_type = other DNA
catcatggaggagcagaagct

SEQ ID NO: 39 - DNA, 37 bp, synthetic construct
  source 1..37: mol_type = other DNA
gcgctctgtgtgcagccatacgacgtaccagattacg

SEQ ID NO: 40 - DNA, 37 bp, synthetic construct
  source 1..37: mol_type = other DNA
tcatgagtcgacactacatacgacgtaccagattacg

SEQ ID NO: 41 - DNA, 37 bp, synthetic construct
  source 1..37: mol_type = other DNA
tatctatcgtatacgccatacgacgtaccagattacg

SEQ ID NO: 42 - DNA, 37 bp, synthetic construct
  source 1..37: mol_type = other DNA
atcacactgcatctgacatacgacgtaccagattacg

SEQ ID NO: 43 - DNA, 37 bp, synthetic construct
  source 1..37: mol_type = other DNA
acgtacgctcgtcatacatacgacgtaccagattacg

SEQ ID NO: 44 - DNA, 37 bp, synthetic construct
  source 1..37: mol_type = other DNA
gcgctctgtgtgcagccatcatggaggagcagaagct

SEQ ID NO: 45 - DNA, 37 bp, synthetic construct
  source 1..37: mol_type = other DNA
tcatgagtcgacactacatcatggaggagcagaagct

SEQ ID NO: 46 - DNA, 37 bp, synthetic construct
  source 1..37: mol_type = other DNA
tatctatcgtatacgccatcatggaggagcagaagct

SEQ ID NO: 47 - DNA, 37 bp, synthetic construct
  source 1..37: mol_type = other DNA
atcacactgcatctgacatcatggaggagcagaagct

SEQ ID NO: 48 - DNA, 37 bp, synthetic construct
  source 1..37: mol_type = other DNA
acgtacgctcgtcatacatcatggaggagcagaagct